(19) United States
(12) Patent Application Publication
(10) Pub. No.: US 2024/0293127 A1
(43) Pub. Date: Sep. 5, 2024

(54) STRETCH RESISTANT EMBOLIC COIL

(71) Applicant: MicroVention, Inc., Aliso Viejo, CA (US)

(72) Inventors: Todd Hewitt, Aliso Viejo, CA (US); Karishma Desai, Aliso Viejo, CA (US); Parker Milhous, Aliso Viejo, CA (US); Tao Lin, Aliso Viejo, CA (US)

(73) Assignee: MicroVention, Inc., Aliso Viejo, CA (US)

(21) Appl. No.: 18/576,356

(22) PCT Filed: Jul. 7, 2022
(86) PCT No.: PCT/US2022/073509

§ 371 Date: Jan. 3, 2024

Related U.S. Application Data
(60) Provisional application No. 63/219,282, filed on Jul. 7, 2021.

(51) Int. Cl. A61B 17/12

(52) U.S. Cl. CPC A61B 17/12154; A61B 17/12113

(57) ABSTRACT

An embolic coil having a stretch resistant member for resisting stretching or unwinding of the embolic coil. A tubular member may be attached to a proximal end of the embolic coil. A stretch resistant member may extend through the tubular member and the embolic coil to be attached at its distal end to the embolic coil. The stretch resistant member may include an enlargement to prevent the stretch resistant member from withdrawing into the tubular member. The stretch resistant member may comprise a one-piece design comprising a tether or a two-piece design comprising a tether in combination with another component such as a filament, a braid, or an eyelet. The stretch resistant member may be wavy to allow slack. The distal end of the stretch resistant member may be attached to the embolic coil by tying a knot, melting into a tip, or the use of adhesives, welding, or soldering.

### RELATED APPLICATIONS

This application claims priority to U.S. Provisional Application Ser. No. 63/219,282 filed Jul. 7, 2021, entitled Centered Coil Knot Configuration, which is hereby incorporated herein by reference in its entirety.

### BACKGROUND OF THE INVENTION

Medical conditions such as aneurysms or similar vascular malformations are often treated endovascularly with small-diameter metallic (most commonly Platinum) embolic coils. These coils are attached to long (˜150-200 cm) delivery systems such that they can be advanced and retrieved through microcatheters. Upon placement of the embolic coil into the aneurysm sac, the implant can be detached from the delivery system using electrolytic, electro-thermal, or mechanical detachment.

### SUMMARY OF THE INVENTION

The present invention is generally directed to an embolic coil having a stretch resistant member to prevent unwanted stretching or unwinding of the embolic coil during delivery through a delivery device such as a catheter. Additionally, the arrangement of the stretch resistant member may allow for improved flexibility, softness, and on-axis torque.

In some example embodiments, a tubular member such as a marker band may be attached to a proximal end of the embolic coil. The tubular member may be comprised of a hypotube. The tubular member may be radiopaque. The inner diameter of an interior lumen of the tubular member may be less than an inner diameter of an interior of the embolic coil.

In some example embodiments, the stretch resistant member may include an enlargement or stopper such as a knot which has a diameter or width which is greater than the inner diameter of the interior lumen of the tubular member to prevent the stretch resistant member from being withdrawn into the tubular member.

In some example embodiments, the stretch resistant member may comprise a one-piece configuration including a tether. The tether may extend through the tubular member and into the interior of the embolic coil. The distal end of the tether may be attached to a distal end of the embolic coil.

In some example embodiments, the stretch resistant member may comprise a two-piece configuration including a tether and a separate component such as a filament, a braid, or an eyelet. The distal end of the tether may be attached to a proximal end of the filament, braid, or eyelet.

In some example embodiment, the stretch resistant member may comprise a tether and a filament attached between a distal end of the tether and a distal end of the embolic coil. The filament may be comprised of a smaller diameter or width than the tether. The connection between the tether and the filament may be encapsulated in adhesive or the like.

In some example embodiments, the stretch resistant member may comprise a tether and a braid. The braid may extend through the embolic coil. The braid may have an outer diameter which is less than the inner diameter of the embolic coil to prevent the braid from contacting the embolic coil. The braid may be formed from a woven filament comprised of various polymeric or metallic materials.

In some example embodiments, the stretch resistant member may comprise a tether and an eyelet. The eyelet may be comprised of a filament formed into a closed loop or into an open loop to have a substantially U-shaped configuration.

In some example embodiments, the stretch resistant member may be attached to the embolic coil by a distal connection. The distal connection may be comprised of a tied knot, an adhesive, welding, soldering, or melting into a distal cap.

The present invention may be directed to a knot design that ensures the connection between the stretch resistant member and the embolic coil is located along a central axis of the embolic coil.

One aspect of the invention may provide a stretch resistant and attachment zone monofilament that runs down the centerline of the device, thus eliminating any off-axis torque caused by terminating the monofilaments on the embolic coil wire.

Another aspect of the invention may provide stretch resistant and attachment zone monofilaments that could be assembled (looped and knotted) outside of the embolic coil, thus eliminating the need to manipulate or ‘open’ the embolic coil up to tie knots.

Another aspect of the invention may provide a smaller diameter, ‘dual strand’ stretch resistance that allows for an increase in tensile strength and relative decrease in the stiffness of the stretch resistant member.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1A is a side view of a pusher including an embolic coil with a stretch resistant member according to one embodiment.

FIG. 1B is a side view of an embolic coil with a stretch resistant member according to one embodiment.

FIG. 1C is a cutaway side view of an embolic coil with a stretch resistant member according to one embodiment.

FIG. 1D is a cutaway side view of an embolic coil with a stretch resistant member being stopped from entering a tubular member according to one embodiment.

FIG. 2A is a side view of a pusher including an embolic coil with a stretch resistant member according to one embodiment.

FIG. 2B is a side view of an embolic coil with a stretch resistant member according to one embodiment.

FIG. 2C is a cutaway side view of an embolic coil with a stretch resistant member according to one embodiment.

FIG. 3A is a side view of an embolic coil with a stretch resistant member according to one embodiment.

FIG. 3B is a cutaway side view of an embolic coil with a stretch resistant member according to one embodiment.

FIG. 4A is a side view of an embolic coil with a stretch resistant member according to one embodiment.

FIG. 4B is a cutaway side view of an embolic coil with a stretch resistant member according to one embodiment.

FIG. 5A is a side view of an embolic coil with a stretch resistant member according to one embodiment.

FIG. 5B is a cutaway side view of an embolic coil with a stretch resistant member according to one embodiment.

FIG. 6A is a side view of an embolic coil with a stretch resistant member according to one embodiment.

FIG. 6B is a cutaway side view of an embolic coil with a stretch resistant member according to one embodiment.

FIG. 7A is a side view of an embolic coil with a stretch resistant member according to one embodiment.

FIG. 7B is a cutaway side view of an embolic coil with a stretch resistant member according to one embodiment.

FIG. 8A is a side view of an embolic coil with a stretch resistant member according to one embodiment.

FIG. 8B is a cutaway side view of an embolic coil with a stretch resistant member according to one embodiment.

FIG. 9A is a cutaway side view of an embolic coil with a stretch resistant member including an enlargement formed from adhesive according to one embodiment.

FIG. 9B is a cutaway side view of an embolic coil with a stretch resistant member including an adhesive-encapsulated knot according to one embodiment.

FIG. 9C is a cutaway side view of an embolic coil with a stretch resistant member including an enlargement according to one embodiment.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Each of the example embodiments shown and described herein is merely an example of a configuration for securing a stretch resistant member to an embolic coil. It should be appreciated that such example embodiments are not meant to be limiting in scope.

The terms coil, embolic coil, microcoil, vaso-occlusive coil and, occlusive coil may be interchangeably used in this specification to refer to an elongated device comprising at least a wire coil suitable for delivering within an aneurysm or other vascular malformation for treatment purposes.

The present invention is generally directed to an embolic coil having a tether that is connected within the embolic coil by an enlargement or stopper near a proximal end of the embolic coil. The enlargement or stopper may be fixed to or part of the tether and may be generally large enough (e.g., width, diameter) that it is prevented from passing proximally through a passage of a tubular member at a proximal end of the embolic coil, thereby preventing part or all of the tether within an interior of the embolic coil from being pulled out. As discussed further below, this arrangement may provide more uniform flexibility/softness to the embolic coil, and torque from the delivery device (e.g., pusher) may be more uniformly or predictably conveyed to the embolic coil due to its axial positioning.

The tether may generally extend from a distal end of a delivery device into a proximal end of the embolic coil and through the tubular member. The tubular member may be located and fixed within an interior of the embolic coil or can be located and fixed partially or fully outside of the embolic coil and to the proximal end of the embolic coil. The tether may be freely longitudinally movable within the tubular member or may be fixed within the tubular member with adhesive. The tubular member may include a passage between its proximal and distal ends that has a diameter that is smaller than the stopper or enlargement. The tubular member may optionally include a radiopaque material to help visualize the proximal end of the embolic coil.

The stopper or enlargement may be a knot formed with the tether. The knot may be formed only with itself and not around other components. The knot may also optionally include adhesive. The stopper or enlargement may also be only adhesive positioned on or around the tether.

The stopper or enlargement may also be a structural component that is separate from the structural wire of the embolic coil. Such structural components may have a passage through which the tether is positioned through and/or the tether may pass around the structural component one or more times. The tether may form a knot around or on one or both sides of the structural component. The adhesive may also be included alone or with the knots to secure the structural component. The structural component may also or alternately be attached via a clamp mechanism that is part of or separate from the structural component. Welding or soldering may also be used to connect the structural component to the tether. The structural component can have a variety of different shapes such as a loop shape, cylinder shape, sphere shape, oval shape, square shape, or rectangular shape.

The tether may terminate at or near the proximal connection point (i.e., the knot, adhesive, or structural member), may terminate at or near a distal end of the embolic coil, or may terminate at locations in between. If the tether is providing stretch resistance to the embolic coil, a tether terminating at the proximal region (or regions proximal of the distal end) of the embolic coil may be connected to a structural component that extends and connects to the distal end of the embolic coil (e.g., a loop, cable, second tether, braid, plurality of links, or similar components). If the tether is connected at or near the distal end of the embolic coil, the tether may connect to a distal end cap or similar structural element on the embolic coil, may form a knot holding it to the embolic coil, may be connected via adhesive to the embolic coil, may be melted to the embolic coil, or similar connection mechanisms may be used. If the tether is connected at or near the distal end of the embolic coil, it may be configured to have tension between both connection points or to have an amount of slack between the connection points such that the tether is wavy when not under tension.

The term stretch resistant member is generally used in this specification to refer to any components, alone or in combination with others, that help resist or prevent an embolic coil to unduly stretch and/or damaged, particularly by forces pulling the embolic coil in a proximal direction. These stretch resistant members may generally be located within an interior of an embolic coil, may be flexible or non-flexible, and may be comprised of polymers, metals, or combinations thereof. The term stretch resistant member may include both tethers and non-tether components.

The term tether may also be referred to as a monofilament and generally refer to an elongated and flexible member that is suitable for use in releasably connecting to an implant, such as an embolic coil. The tether may comprise any of the various types of tethers known in the art, such as but not limited to a polymer monofilament composed of various polymers (e.g., PET, Engage, polypropylene, and the like). The tether may also function as part of a detachment mechanism by being severed by a mechanism of a delivery device (e.g., by cutting, melting, or the like).

It can be beneficial for embolic coils to be especially flexible or “soft” in order to prevent placing undue forces on the walls while being advanced out of a delivery catheter. However, this flexibility and softness may result in a more delicate embolic coil structure that can be prone to damage. Thus, such coils may suffer from various shortcomings such as stretching or unwinding during delivery. By incorporating a stretch resistant member into the embolic coil design, coils can be made even softer while reducing or eliminating the likelihood of stretching or unwinding.

To help maintain a desirable amount of tension in the embolic coil, the tether may sometimes be connected to both the distal and proximal ends of the embolic coil (either directly or indirectly via other components). However, these connection points may otherwise change certain performance characteristics for the embolic coil during delivery, particularly at or near the connection points of the tether to the embolic coil. For example, the embolic coil may have reduced or nonuniform flexibility/softness near the proximal connection point. In examples where the tether is tied to the wire forming the embolic coil (i.e., a side of the embolic coil), off-axis torque may sometimes result between the pusher and the embolic coil since the tether extends towards/away from a side of the interior of the embolic coil. This nonuniform flexibility and off-axis conveyance of torque can, in some cases, result in changes in how the embolic coil behaves as it exits a delivery catheter (e.g., changes in “kickback”) and/or changes in how movement of the delivery device is imparted to portions of the embolic coil that have been pushed out of the delivery catheter.

The connection techniques of the present invention may eliminate the need to connect the tether around a portion of the embolic coil's wire (i.e., directly to a side of the embolic coil) and, in some cases, may better center the tether within the embolic coil. For example, the stopper or enlargement may generally position the tether near a central longitudinal axis of the embolic coil and in a manner that is free from direct contact with or connection to a side of the embolic coil. Hence, greater uniformity of flexibility/softness and better, on-axis conveyance of torque from the delivery device (e.g., pusher) to the embolic coil may be achieved.

Although not shown, the embolic coil 100 may be releasably attached to a distal end of a pusher and, upon delivery to a target location, detached from the pusher. The manner of detachment of the embolic coil 100 from the pusher may vary in different embodiment and may comprise any manner of detachment known in the art.

A heater coil may be utilized for detaching the implant such as an embolic coil 100. The tether may pass into an interior of a pusher, through a heater coil, and then be fixed to a structural coil or other component of the pusher. Activation of the heater coil by the operator may cause the tether to break or melt so as to release the implant.

Additional non-limiting examples of detachment mechanisms which may be used with the present invention, including any of the embodiments shown or described herein, may include those shown and/or described in U.S. Pat. Nos. 10,980,544, 10,265,077, 9,717,500, 9,561,125, 8,460,332, 8,192,480, 8,182,506 and U.S. Publication Nos. US20060200192, US20090062812, US20090163780, US20100268204, US20110301686, US20150289879, all of which are hereby incorporated by reference.

Specific example embodiments are described further below. However, it should be understood that any of the features from any of the embodiments can be mixed and matched with each other in any combination. Hence, the present invention should not be restricted to only these embodiments, but any broader combination thereof.

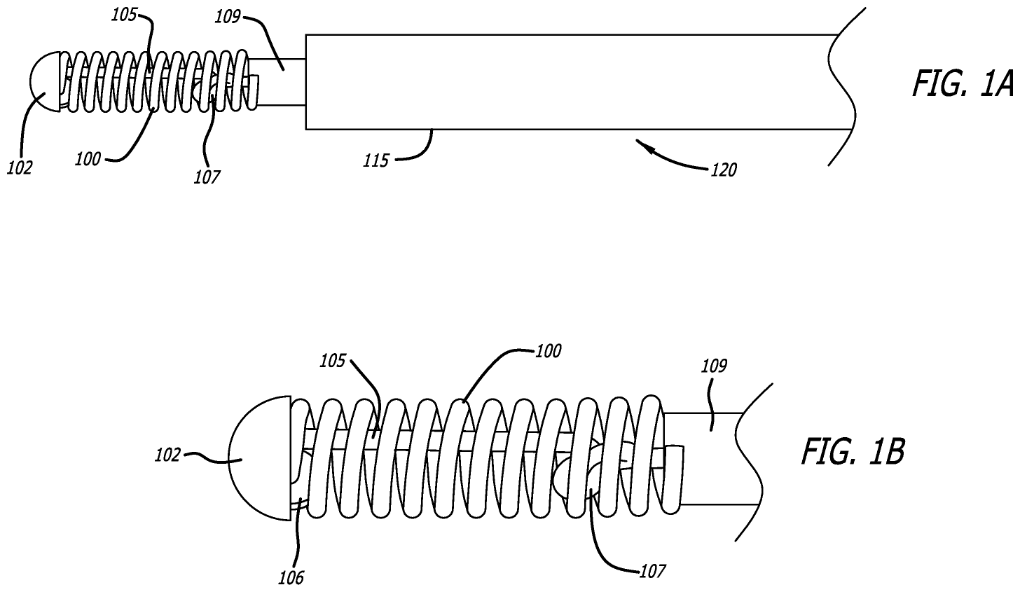

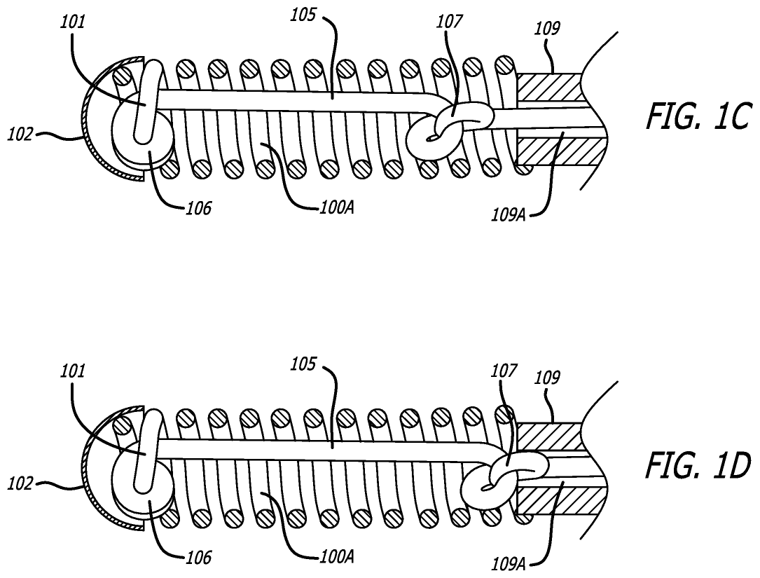

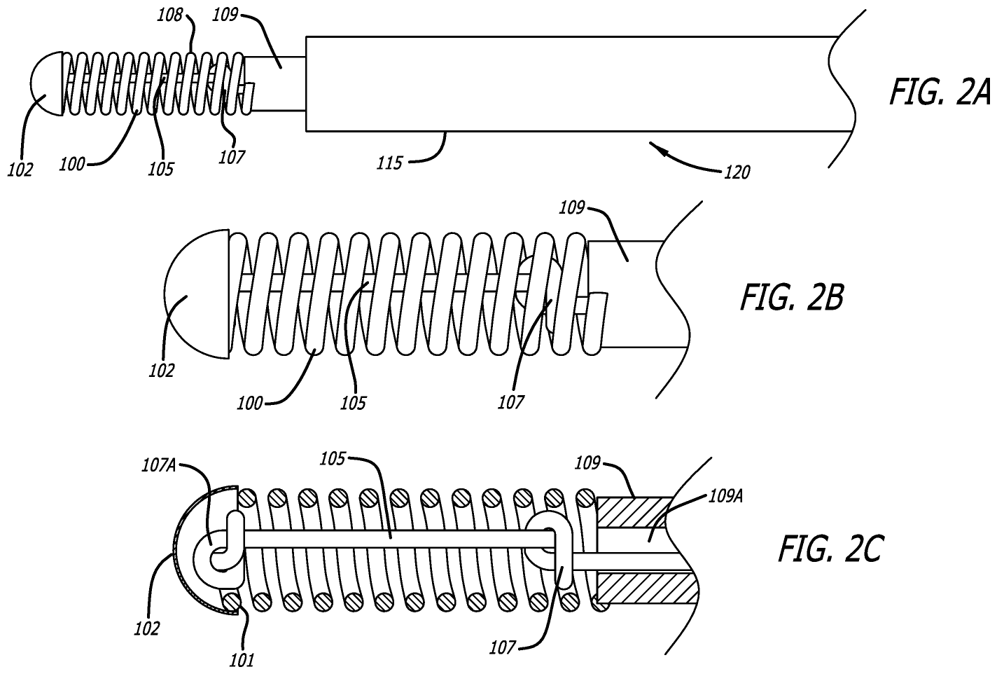

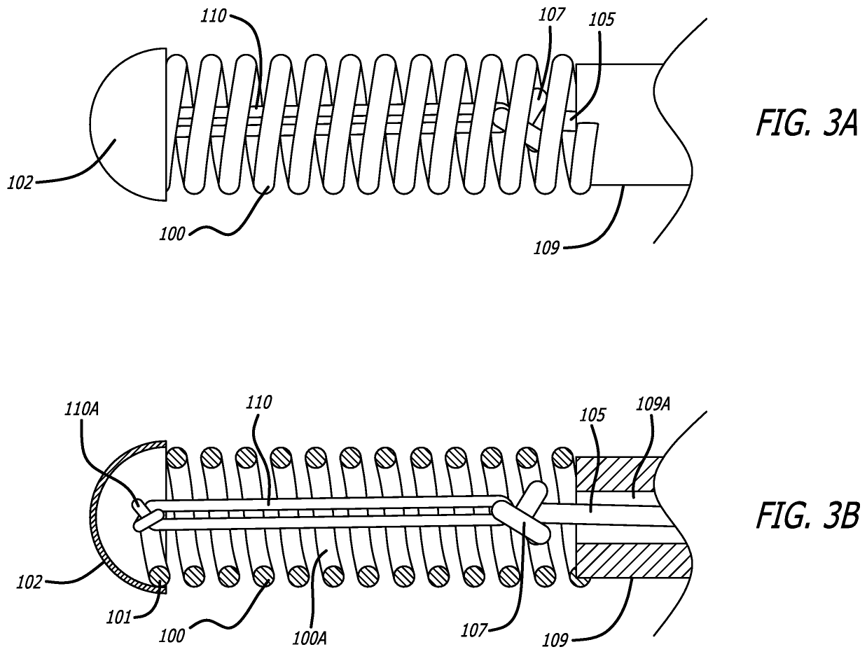

FIGS. 1A and 2A illustrate an example embodiment of a stretch resistant embolic coil device 120 having a delivery device 115, an embolic coil 100, and a stretch resistant member 105. It has been found that occlusive coils may be prone to stretching or unwinding either during delivery or during repositioning within the body. The example embodiments of stretch resistant members 105 shown and described herein may be utilized to reduce or eliminate the likelihood of such undesirable situations.

The figures illustrate various example embodiments of an embolic coil 100 formed from a wound wire 101. It should be appreciated that the embolic coils 100 shown in the figures are merely for exemplary purposes, and thus the configuration, shape, and size of the embolic coils 100 should not be construed as limited by the example embodiments shown in the figures. For example, for clarity and simplicity, the embolic coils 100 shown in the figures have a looser winding. It should be appreciated that, in other embodiments, the embolic coils 100 may have tighter windings than the example embodiments shown in the figures. Additionally, while the figures illustrate coils 100 being comprised of a single filar configuration, multi-filar configurations may be utilized in other embodiments, including but not limited to bifilar configurations, trifilar configurations, and so on.

It should also be appreciated that the example embodiment illustrated in the figures are not drawn to scale. The example embodiments of the present invention shown in the figures are drawn in a shortened view for better clarity. The example embodiments of embolic coils, for example, are not drawn to scale and could be longer, shorter, wider, and/or narrower than shown in the figures.

FIGS. 1A and 2A illustrate example embodiments of a delivery system including a catheter 115 and an embolic coil 100 being delivered through the catheter 115. It should be appreciated that the type of catheter 115 or other delivery system utilized may vary in different embodiments, and thus the present invention should not be construed as limiting in scope to any particular configuration or manner of delivery shown in the figures. Generally, the embolic coil 100 may be delivered through an internal lumen of a catheter 115, with the catheter 115 being withdrawn after delivery of the embolic coil 100 to a target location within a patient's body, such as an aneurysm. Alternatively, in some embodiments, the embolic coil 100 may instead be pushed out of the distal end of the catheter 115.

Various embodiments of the present invention may comprise an embolic coil 100 including one or more wires 101 wound or shaped into a tubular coil shape, and one or more (e.g., a one-piece or two-piece) stretch resistant members 105 within an interior 100A of the tubular coil shape. The use of the stretch resistant member 105 may reduce the fragility of the embolic coil 100, even if the embolic coil 100 is particularly soft. When tension is applied, the stretch resistant member 105 may bear the axial load, thus making it more difficult to stretch or unwind the embolic coil 100.

FIGS. 1A-1C illustrate an example embodiment of an embolic coil device which may be comprised of an embolic coil 100 including a stretch resistant member 105 extending at least partially through an interior 100A thereof. As shown in FIG. 1A, the embolic coil 100 may be delivered (e.g., by being pushed by a pusher) through an internal lumen of a catheter 115 or other delivery device. The embolic coil 100 may include a stretch resistant member 105 extending at least partially therethrough which reduces or eliminates the likelihood of the embolic coil 100 stretching or unwinding. In an example embodiment, the stretch resistant member 105 may comprise a tether 105A which may be severed or melted in various manners to detach the embolic coil 100 from the pusher or other delivery device.

FIGS. 1B and 1C show a closer view of an example embodiment of an embolic coil 100. As shown, the embolic coil 100 may comprise one or more wires 101 which are wound into an embolic coil shape. As previously mentioned, the windings of the wire(s) 101 forming the embolic coil 100 may be tighter than is shown in the figures. Additionally, while only a single wound wire 101 (i.e., a single filar configuration) is shown, multiple wound wires 101 (i.e., a multi-filar configuration) may be utilized in some embodiments. The embolic coil 100 may include an interior 100A defined as the internal space within the windings of the embolic coil 100.

As best shown in FIG. 1C, a stretch resistant member 105 may be attached to a position at or near a distal end of the embolic coil 100. FIG. 1C illustrates that the stretch resistant member 105 may be attached to the embolic coil 100 by a distal connection 106, e.g., a knot tied around a distal winding of the wire 101 of the embolic coil 100. It should be appreciated that the stretch resistant member 105 may be attached to the embolic coil 100 at other locations and by other types of distal connections 106, such as discussed in more detail below with respect to other embodiments. As a non-limiting example, the distal connection 106 of the stretch resistant member 105 may be tied not to the distal end of the embolic coil 100 as shown in FIG. 1C, but instead to a position that is more set back towards the proximal end of the embolic coil 100 in some example embodiments.

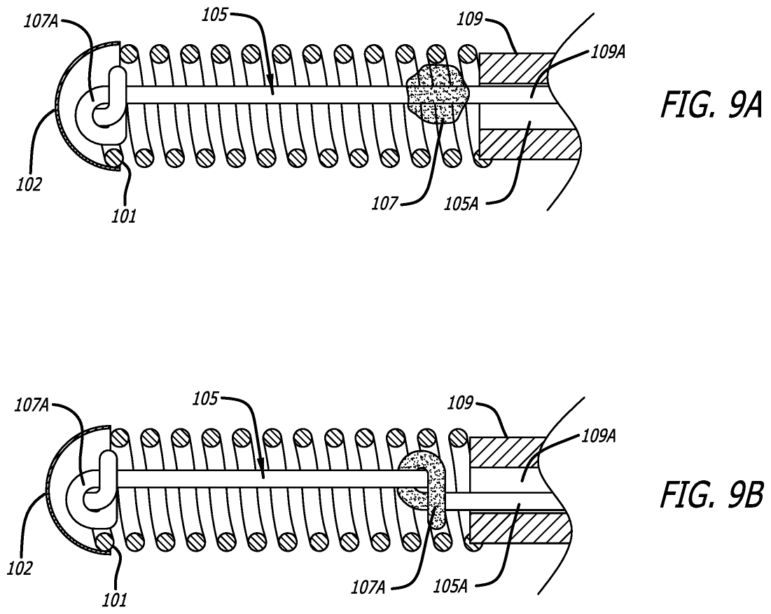

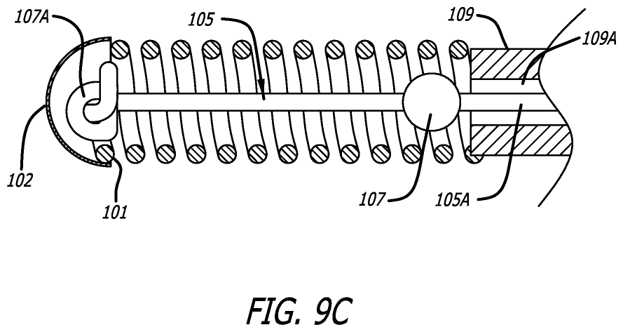

With reference to FIGS. 1C and 1D, it can be seen that the stretch resistant member 105 may comprise an enlargement 107 that functions as a stopper to prevent withdrawal of the stretch resistant member 105 into the tubular member 109 and is positioned proximally with respect to the distal end of the stretch resistant member 105. The enlargement 107 may comprise a knot such as shown in FIGS. 1C and 1D. In other embodiments such as shown in FIG. 9C, the enlargement 107 may comprise various projections or other devices, such as beads, balls, or the like, that will prevent the stretch resistant member 105 from being pulled back into the tubular member 109. Thus, the enlargement 107 may, in some example embodiments, comprise a separate device that is attached to the stretch resistant member 105, such as by an adhesive, soldering, or other methods such as shown in FIG. 9B. In some embodiments, the enlargement 107 may comprise a ball of adhesive or the like such as shown in FIG. 9A.

In example embodiments, the enlargement 107 may not be directly connected to any portion of the embolic coil 100. In some such example embodiments, the enlargement 107 may not contact any portion of the embolic coil 100. Thus, the enlargement 107 may not be fixed, looped, knotted, or otherwise secured or in contact with any portion, such as a wire 101, of the embolic coil 100. In such example embodiments, the enlargement 107 may freely float within the interior 100A of the embolic coil 100, though the enlargement 107 may be prevented from exiting the interior 100A of the embolic coil 100 due to its size being greater than that of an interior lumen 109A of the tubular member 109 attached to the proximal end of the embolic coil 100.

The enlargement 100 may be positioned along a central longitudinal axis extending through the embolic coil 100 or may be axially offset from the central longitudinal axis of the embolic coil 100. The enlargement 107 may be positioned entirely within the interior 100A of the embolic coil 100. The enlargement 107 may be movable with respect to of the embolic coil 100. In some example embodiments, the enlargement 107 may be secured to the embolic coil 100, such as at or near its proximal end.

In the embodiment shown in FIGS. 1A-1D, the enlargement 107 may comprise a freely-moving knot such that the enlargement 107 “floats” within the interior 100A of the embolic coil 100. The type of knot may vary in different embodiments and thus should not be construed as limited by the example knot shown in the figures. Any type of knot known in the art may be utilized.

It can be best seen in FIGS. 1B and 1C that a tubular member 109 may be secured to a proximal end of the embolic coil 100. The manner by which the tubular member 109 and coil 100 are attached may vary in different embodiments, and may include, e.g., use of adhesives, welding, and the like. The tubular member 109 will generally include an internal lumen 109A that is of a smaller diameter than the enlargement 107 such as shown in FIG. 1C. Thus, the enlargement 107 may be prevented from entering into the internal lumen 109A of the tubular member 109 by its width or diameter, and may instead catch onto the distal end of the tubular member 109 if retracted such as shown in FIG. 1D. Such a configuration ensures that the stretch resistant member 105 may not be pulled back into the tubular member 109.

In an example embodiment, the stretch resistant member 105 may function as both a stretch resistant member and a detachment tether (a “one-piece design”) in some example embodiments. In other embodiments as discussed herein, the stretch resistant member 105 may instead comprise a “two-piece design” in which the stretch resistant member 105 comprises both a detachment tether 105A and a separate structure, such as another tether, a filament 110, a braid 108, and/or an eyelet 111, that is attached to the detachment tether 105A in various manners.

Generally, the stretch resistant member 105 may extend through the tubular member 109 and into the catheter 115. A proximal end of the stretch resistant member 105 may be attached either to the embolic coil 100, to the tubular member 109, to a pusher, or to the catheter 115 using various methods known in the art for securing an elongated member such as a tether to another structure. In this manner, the stretch resistant member 105 may be anchored at its proximal end.

Continuing to reference FIGS. 1A-1D, it can be seen that a cap 102 may be positioned over the distal end of the embolic coil 100. The cap 102 may comprise a melted polymer, metal, or metal alloy formed into a semi-spherical shape such as described below. In other embodiments, the cap 102 may comprise a separate structure that may be attached in various manners to the distal end of the embolic coil 100.

Put differently, instead of securing the stretch resistant member 105 to the wire 101 of the primary wind of the embolic coil 101 (i.e., an “outside knot”), the material forming the stretch resistant member 105, such as a polymer, may be formed into a knot which is completely inside the embolic coil 100 (i.e., an “inside knot”) and which functions as an enlargement 107. The proximal end of the embolic coil 101 (in the current as well as proposed configurations) may comprise a tubular member 109 such as a hypotube with a relatively small inner diameter. The type of material used for the tubular member 109 may be composed of a radiopaque material such as but not limited to gold, platinum, and tantalum, or may be composed of nonradiopaque materials including biocompatible metals, polymers, and the like. This “inside knot” enlargement 107 would be too large in diameter to be pulled through the tubular member 109 and would therefore serve the same purpose a knot tied to the proximal end of the embolic coil. Adhesive could also be added to more securely fix the enlargement 107 to the inside 100A of the embolic coil 100.

The manner by which the embodiment shown in FIGS. 1A-1C is manufactured may vary in different embodiments. In one example embodiment, the illustrated embodiment may be assembled by tying the stretch resistant member 105 into a knot (at what will later become the proximal end) before pulling it through the linearized metal coil. This configuration partially addresses the above concerns as the proximal knot may be centered within the embolic coil, and the embolic coil need not be opened to tie the proximal knot.

FIGS. 2A-2C illustrate another example embodiment of a stretch resistant embolic coil device 120 which may be comprised of an embolic coil 100 including a stretch resistant member 105 extending therethrough. Such an embodiment is similar to the embodiment previously shown and described in FIGS. 1A-1C, except that the distal end of the stretch resistant member 105 is not tied to the embolic coil 100. Instead, the stretch resistant member 105 of the example embodiment shown in FIGS. 2A-2C may include a distal connection 106 comprised of an adhesive which encapsulates and secures the distal end of the stretch resistant member 105 to the embolic coil 100. In the example embodiment shown in FIGS. 2A-2C, the distal connection 106 may comprise a knot that is secured to the embolic coil 100 not by being tied to the embolic coil 100 as in FIGS. 1A-1C, but by other methods such as but not limited to adhesives or the like.

Both of the embodiments shown in FIGS. 1A-1C and 2A-2C illustrate a pair of knots, with the distal knot being utilized as a distal connection 106 to the embolic coil 100 and the proximal knot being utilized as an enlargement 107 to prevent the stretch resistant member 105 from being withdrawn into the catheter 115. While the figures illustrate that both knots comprise a similar size and configuration, it should be appreciated that, in some embodiments, the respective sizes of the knots may be different.

Continuing to reference FIG. 2C, it can be seen that both knots (the knot forming the distal connection 106 and the knot forming the enlargement 107) may be positioned within the interior 100A of the embolic coil 100. Both knots may be aligned with each other along a longitudinal central axis of the embolic coil 100. In other embodiments, the knots may not be linearly aligned such as shown in the figures. Further, it should be appreciated that one or both of the knots may in some embodiments be offset with respect to the longitudinal central axis of the embolic coil 100. While not shown, in some embodiments, additional knots may be included (e.g., three, four, or more knots).

The distal knot may be positioned at or near the distal end of the stretch resistant member 105 so as to form a distal connection 106. The proximal knot, which forms the enlargement 107, may be inset with respect to the distal end of the stretch resistant member 105 towards its proximal end such as shown in FIG. 2C. The distance between the first and second knots may vary in different embodiments and thus should not be construed as limited by the example embodiments shown in the figures. Additionally, the size of each of the knots as compared to the inner diameter of the embolic coil 100 and to each other may vary in different embodiments and similarly should not be construed as limited by the example embodiments shown in the figures.

The distal knot forming the distal connection 106 may be secured to the embolic coil 100 in various manners. As previously discussed with respect to the embodiment shown in FIGS. 1A-1C, the distal connection 106 may be tied to a wire 101 of the embolic coil 100. As a further example as shown in FIG. 2C, the distal connection 106 may be secured to the embolic coil 100 by adhesives, soldering, or the like. In one example embodiment, the distal connection 106 may be encased in adhesive and secured to the wire 101 of the embolic coil 100 at or near its distal end.

Continuing to reference the example embodiment shown in FIGS. 2A-2C, the knots forming the distal connection 106 and enlargement 107 may be tied either before or after pulling the stretch resistant member 105 through the embolic coil 100. In the latter case, the knots may be inserted (e.g., pushed) inside the distal tip of the embolic coil 100 after being tied. In either case, the knot forming the distal connection 106 may be encapsulated in adhesive which also secures the stretch resistant member 105 to the wire 101 of the embolic coil 100. In some embodiments, the distal connection 106 may not be a knot, but may instead comprise a distal end of the stretch resistant member 105 (e.g., a tether 105A) that is attached or secured to the embolic coil 100 by various manners including but not limited to encapsulation in adhesive.

FIGS. 3A-3B illustrate another example embodiment of a stretch resistant embolic coil device 120 which may be comprised of an embolic coil 100 including a two-piece configuration of a stretch resistant member 105 which comprises both a tether 105A and a filament 110 that may be secured by various manners to a distal end of the tether 105A. Thus, the stretch resistant member 105 may comprise both a first detachment tether 105A which extends through the tubular member 109 into the embolic coil 100 and a second linkage/connection tether such as a filament 110 or the like which is attached between a distal end of the first detachment tether 105A and a position at or near a distal end of the embolic coil 100.

As best shown in FIG. 3B, it can be seen that a distal end of the first stretch resistant member 105 may include an enlargement 107 which is of a greater diameter than that of the tubular member 109 from which the tether 105A extends. The enlargement 107 may comprise an eyelet, clamp, clasp, bead, ball, or knot having an open loop or other opening through which the filament 110 may be threaded.

The filament 110 may be attached between the distal end of the tether 105A and a position at or near the distal end of the embolic coil 100. In the example embodiment shown in the figures, it is illustrated that the filament 110 may be looped through the enlargement 107, such as through an opening thereof. However, various other means may be utilized in different embodiments to attach the filament 110 to the distal end of the tether 105A.

The second piece of the stretch resistant member may comprise a tether, filament 110, or the like which is formed into an elongated loop such as shown in FIG. 3B. Thus, the filament 110 may be looped through the enlargement 107 at the proximal end of the filament 110 and anchored or secured to or near the distal end of the embolic coil 100 at the distal end of the filament 110. The filament 110 may comprise various materials, such as but not limited to various polymers.

In the embodiment shown in FIG. 3B, it can be seen that the distal end of the filament 110 may form a distal connection 106 with the embolic coil 100, such by tying the distal end of the filament to the embolic coil 100. More specifically, the distal end of the filament 110 may be tied to a wire 101 of the embolic coil 100, such as the distal-most winding of the wire 101 forming the embolic coil's 100 primary wind as illustrated. However, it should be appreciated that the distal end of the filament 110 may be tied to various other locations on the embolic coil 100 other than its distal most end in some embodiments. Further, it should be appreciated that manners other than tying may be utilized to secure the distal end of the second stretch resistant member 110 to the embolic coil 100 as discussed below.

Thus, FIGS. 3A-3B illustrate an example two-piece embodiment of the stretch resistant member 105, 110. In such an embodiment, the distal end of a detachment tether 105A, such as a polymer, may be knotted in such a way as to leave an open loop to thread a separate filament 110, such as a polymer, therethrough (much like the “eye” of a needle). The filament 110 can be looped through this “eye” in the tether 105A proximally and the two strands can be tied together and encapsulated in adhesive distally. Most of these steps (tying of the knot and looping the filament 110 through the knot) may be completed prior to pulling the stretch resistant member 105 through the linearized coil 100. Such an embodiment retains the advantages of the embodiment shown in FIGS. 2A-2B while also allowing for separate filaments to form the stretch resistant member 105.

For example, as shown in FIG. 3B, the filament 110 may comprise a different width or diameter than the tether 105A, which may allow the filament 110 to be more flexible than the tether 105A. However, in some embodiments, the reverse configuration may be utilized (e.g., the filament 110 may be larger and less flexible than the tether 105A). In other embodiments, both the filament 110 and the tether 105A may comprise the same or substantially the same size and/or flexibility.

With reference to the example embodiments shown in FIGS. 2A-3B, it should be appreciated, as previously mentioned, that the sizes of the respective tether 105A and filament 110 may vary in different embodiments. As a non-limiting example, the gauge of the tether 105A may be between 1.5-2.5 thousands, such as 2.2 thousands. The gauge of the filament 110 may be between 0.5-1.5 thousands, such as 0.9 thousands. The filament 110 may comprise various materials such as but not limited to PET, propylene, nylon, or the like.

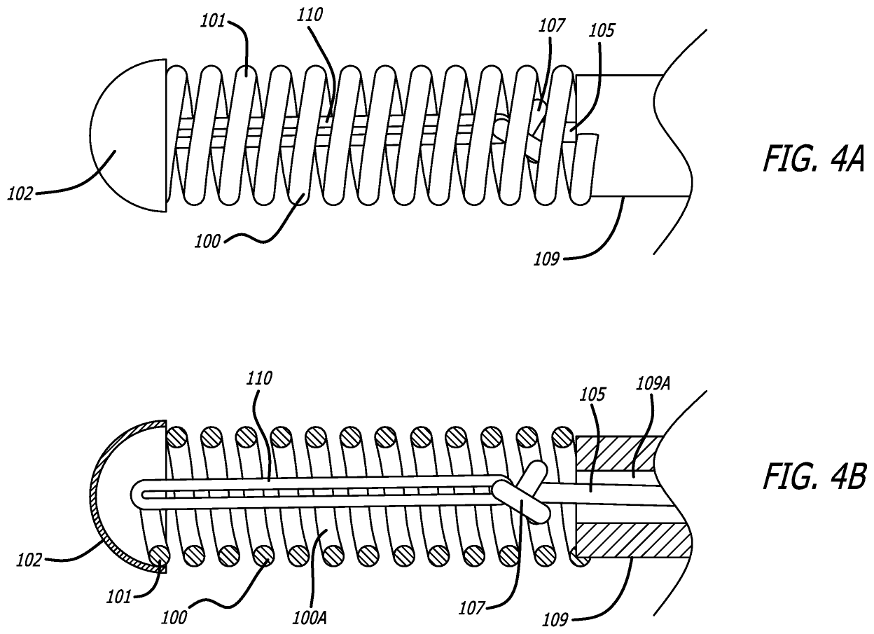

FIGS. 4A-4B illustrate another example embodiment of a stretch resistant embolic coil device 120 which may be comprised of an embolic coil 100 including a two-piece configuration of a stretch resistant member 105 which comprises both a tether 105A and a filament 110 that may be secured by various manners to a distal end of the tether 105A. In such an example embodiment, the filament 110 may comprise an elongated loop composed of various biocompatible polymers known in the art.

As shown in FIGS. 4A-4B, such an example embodiment may be similar to the example embodiment of FIGS. 3A-3B except with respect to the distal connection 106 of the filament 110. Rather than being tied to the embolic coil 100 as in FIGS. 3A-3B, FIGS. 4A-4B illustrate an example embodiment in which the distal connection 106 of the filament 110 is formed by melting the filament into a distal cap 102 or ball that is fused or otherwise adhered against the distal end of the embolic coil 100. As the polymer at the distal end of the filament 110 is melted to a cap 102 such as a ball at the distal tip, this distal tip design may be easier to form as it does not require the tying of a knot.

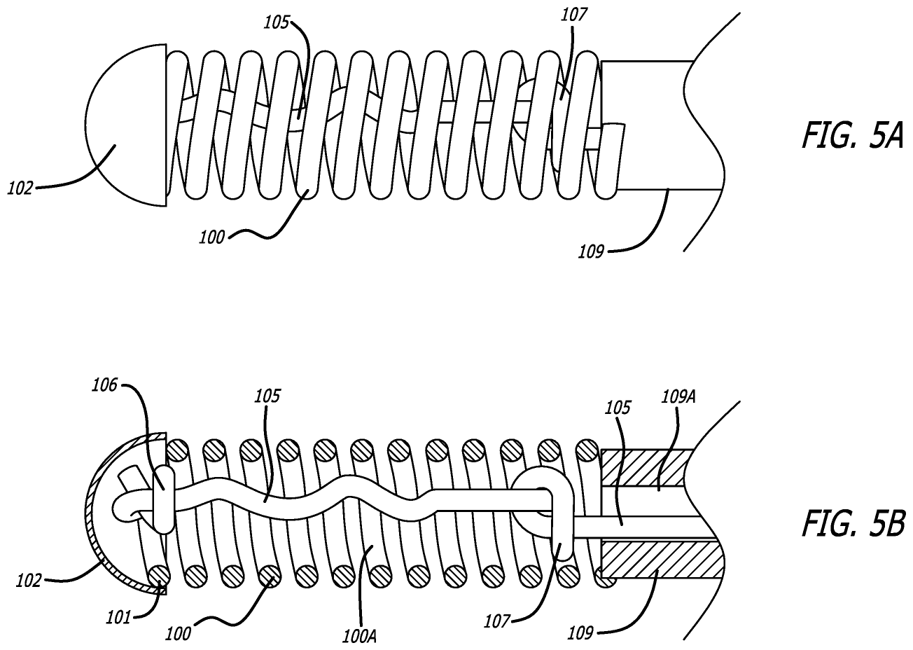

FIGS. 5A-5B illustrate another example embodiment of a stretch resistant embolic coil device 120 which may be comprised of an embolic coil 100 including a one-piece stretch resistant member 105 comprising a tether 105A having both an enlargement 107 and a distal connection 106 to the embolic coil 100. It should be appreciated, however, that the following features described with respect to the example embodiment shown in FIGS. 5A-5B may be applied to any of the preceding or subsequent example embodiments shown and described herein, including both one-piece and two-piece stretch resistant member 105 configurations.

Continuing to reference FIGS. 5A-5B, it can be seen that the stretch resistant member 105 may comprise a tether 105A which extends from the lumen 109A of a tubular member 109 such as a hypotube. The stretch resistant member 105 includes an enlargement 107 which may comprise a knot that is larger than the size of the lumen 109A of the tubular member 109 so as to prevent the stretch resistant member 105 from being pulled into the tubular member 109. The stretch resistant member 105 may include a distal connection 106 comprised of a knot tied to a wire 101 of the embolic coil 100 as shown in FIG. 5B, though the distal connection 106 may alternatively comprise any of the other types of distal connections 106 shown or described herein, such as but not limited to use of an adhesive or the like.

As best shown in FIG. 5B, the stretch resistant member 105 may have extra slack by being wavy or helical. Regardless of distal or proximal securement techniques, the stretch resistant member 105 may be slightly “wavy” or helical to provide extra “slack” and mitigate over-tensioning during assembly or subsequent exposure to heat. These “waves” can be added by helically winding the stretch resistant member 105 around a wire or other polymer prior to assembly and then removing the extra wire or polymer after the stretch resistant member 105 has been pulled through the linearized metallic coil 100. Another method for creating these “waves” would be leaving the portion of the stretch resistant member 105 intended to extend within the interior 100A of the embolic coil 100 longer than the embolic coil 100 and then pushing the extra length of the stretch resistant member 105 into the embolic coil 100 from the distal end after the distal connection 106 is formed (e.g., by being tied). In either case, the extra slack ensures that the embolic coil 100 is not under compression after assembly.

Continuing to reference FIGS. 5A-5B, the illustrated waviness of the stretch resistant member 105 may allow some slack for the stretch resistant member 105. The waviness may be imparted to the stretch resistant member 105 by various manners. In one example embodiment, the stretch resistant member 105 may be wound at a very low/loose pitch over a fixture such as a mandrel so as to have a very slightly coiled shape. In another example embodiment, the stretch resistant member 105 intended to extend within the embolic coil 100 may be cut just slightly longer than the length of the embolic coil 100 prior to being bonded within the embolic coil such that the stretch resistant member 105 may relax into the illustrated wavy configuration. As a non-limiting example, if the embolic coil 100 is 9 cm in length, the portion of the stretch resistant member 105 extending through the interior 100A of the embolic coil 100 may be 10 cm in length such that, after the stretch resistant member 105 is secured within the embolic coil 100, it may buckle into the wavy shape.

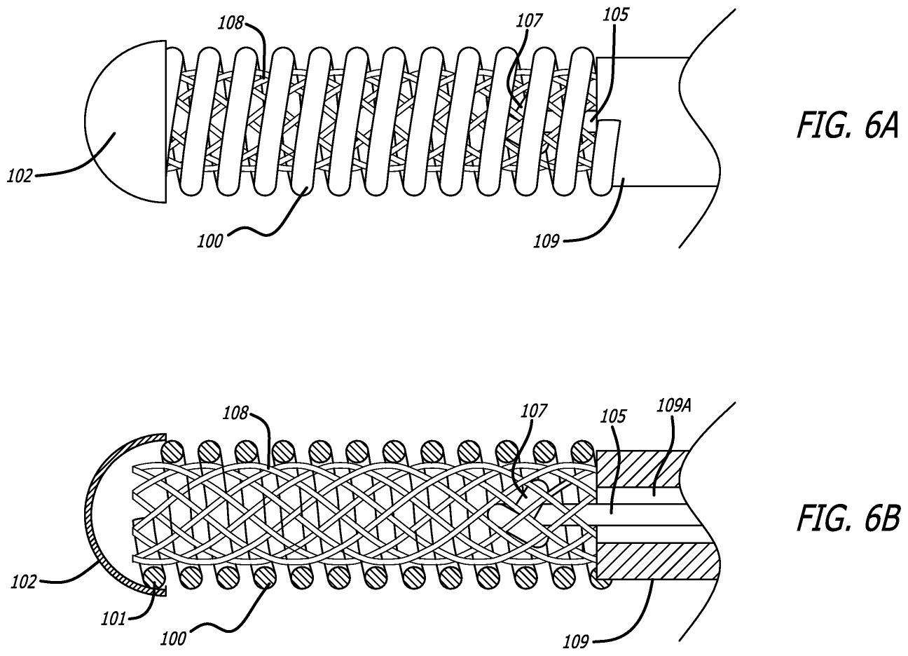

FIGS. 6A-6B illustrate another example embodiment of a stretch resistant embolic coil device 120 which may be comprised of an embolic coil 100 including a two-piece stretch resistant member 105 comprising a tether 105A having an enlargement 107 and a braid 108 positioned within the interior 100A of the embolic coil 100. Thus, in the example embodiment shown in FIGS. 6A-6B, the stretch resistant member 105 may comprise both a tether 105A which extends from a tubular member 109 such as a hypotube and a braid 108 which extends along all or part of the length of the interior 100A of the embolic coil 100.

As best shown in FIG. 6B, the braid 108 may be formed from strands of one or more filaments or the like formed into a braided structure. Various types of materials may be utilized to form the braid 108, including but not limited to various polymers, metals, and/or metal alloys. The braid 108 may be tubular as shown in the figures or may comprise various other shapes.

In some example embodiments, the outer diameter of the braid 108 may be less than the inner diameter of the embolic coil 100 such that the braid 108 does not directly contact the embolic coil 100. Such a configuration may ensure that minor compression of the embolic coil 100 does not result in the braid 108 being locked against the embolic coil 100. Thus, it may be desirable to leave a little clearance between the outer diameter of the braid 108 and the inner diameter of the embolic coil 100. However, in some embodiments, the outer diameter of the braid 108 may be substantially similar to the inner diameter of the embolic coil 100 such that the outer diameter of the braid 108 may rest against the inner diameter of the embolic coil 100.

Further, the winding pattern of the braid 108, the pore-size of the braid 108, and/or the picks-per-inch of the braid 108 may vary in different embodiments and should not be construed as limited by the example embodiment shown in the figures.

Continuing to reference FIG. 6B, a distal end of the braid 108 may be secured against the embolic coil 100 in various manners. In one example embodiment such as shown in the figures, the distal end of the braid 108 may be encapsulated in an adhesive such as glue or, if metallic, may be welded to the embolic coil 100 (e.g., to a wound wire 101 or primary wind of the embolic coil 100). In some embodiments in which a separate cap 102 is secured to a distal end of the embolic coil 100, the distal end of the braid 108 may be secured in various manners to the cap 102. The proximal end of the braid 108 may be secured to the embolic coil 100, to the tubular member 109, and/or to the tether 105A (e.g., by a knot). In some embodiments, the braid 108 may be secured to the enlargement 107 of the tether 105A.

The gauge of the filament forming the braid 108 may vary in different embodiments. By way of non-limiting example, the gauge of the filament forming the braid 108 may be between 0.5-1.5 thousands, such as 0.9 thousands. The gauge of the tether 105A may be larger in some embodiments, such as but not limited to between 1.5-2.5 thousands, such as 2.2 thousands. The material of the filament forming the braid 108 may also vary and may include as non-limiting examples PET, propylene, nylon, or the like.

This braid 108 of the stretch resistant member 105 may achieve a similar effect as the wavy design shown in FIGS. 5A-5B. This braid 108 may be comprised of polymer or even very small diameter metallic materials such as platinum (same material as the embolic coil 100 but smaller diameter). In the latter case, the distal end of the braid 108 may be attached to the primary wind of the embolic coil 100 by laser welding the materials together. One option for proximal securement, as shown in the figures, may be encapsulating the braid 108 and detachment tether 105A in adhesive. The braid 108 may provide additional strength (due to using multiple strands) but also allow for some axial flexibility (to ensure the embolic coil 100 isn't under tension after assembly). If the stretch resistant member 105 is constructed from platinum or other metallic strands, it can be placed into the embolic coil 100 prior to secondary heat set. In this case, both the stretch resistant member 105 and coil 100 may be heat set into the same or a similar shape.

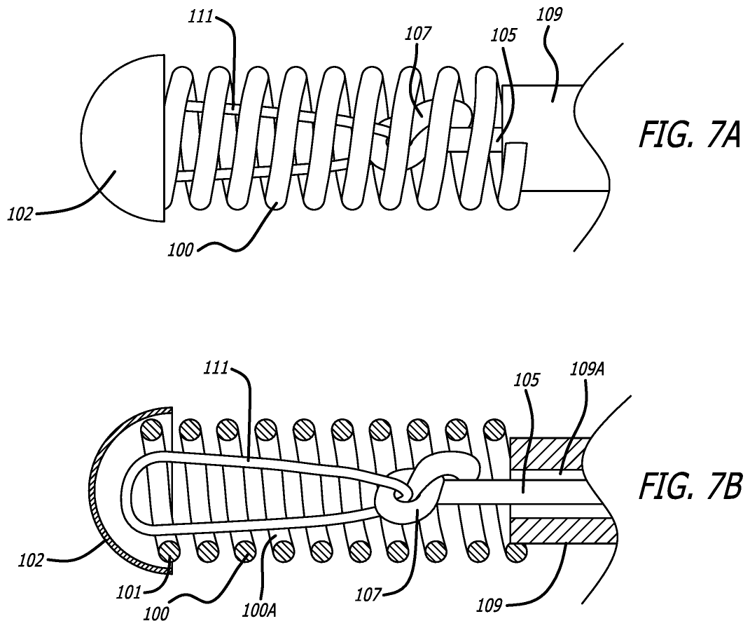

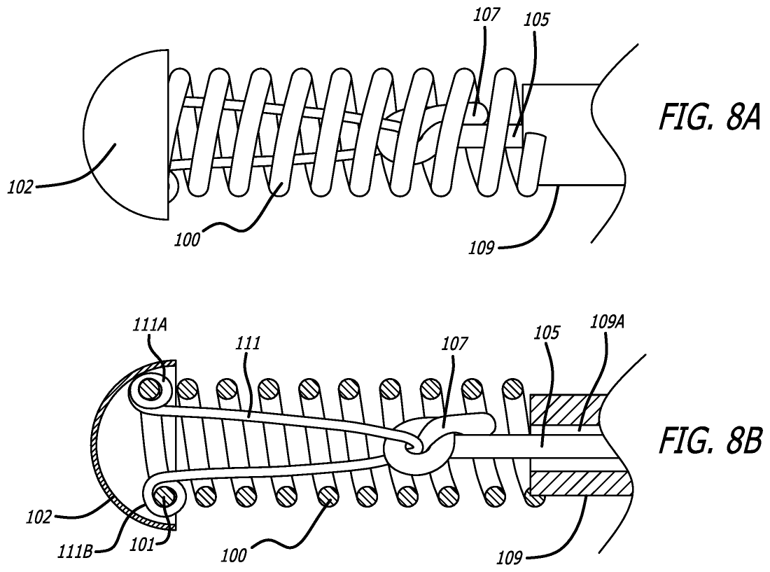

FIGS. 7A-7B and 8A-8B illustrate example embodiments of a stretch resistant embolic coil device 120 which may be comprised of an embolic coil 100 including a two-piece stretch resistant member 105 comprising a tether 105A having an enlargement 107 and an eyelet 111 connected between the distal end of the tether 105A and the distal end of the embolic coil 100. FIGS. 7A-7B illustrate an example embodiment in which the distal end of the eyelet 111 may be melted or adhered with adhesive to the distal end of the embolic coil 100 or to a cap 102. FIGS. 8A-8B illustrate an example embodiment in which the eyelet 111 comprises an open loop having a substantially U-shaped configuration which is secured to the distal end of the embolic coil 100 by a pair of eyelet connections 111A, 11B such as tied knots.

As shown in FIGS. 7A-8B, an eyelet 111 may be attached between the distal end of the tether 105A and a distal region of the embolic coil 100. The eyelet 111 may comprise various polymeric or metallic (including alloys) materials known in the art. The eyelet 111 may comprise a smaller outer diameter than the tether 105A such as shown in the figures. However, in other embodiments, the outer diameter of the eyelet 111 may be greater than or equal to that of the tether 105A.

As best shown in FIGS. 7B and 8B, the proximal end of the eyelet 111 may be attached to the distal end of the tether 105A. In the illustrated example embodiments, it can be seen that the proximal end of the eyelet 111 may be looped through an opening in the enlargement 107 (e.g., knot) at the distal end of the tether 105A. However, various other manners may be utilized for securing the eyelet 111 to the tether 105A, including encapsulation in an adhesive such as glue, tying of knots, clamps, clasps, brackets, welding, soldering, and the like.

With reference to FIG. 7B, it can be seen that a distal end of the eyelet 111 may be secured to the embolic coil 100. In such an embodiment, the eyelet 111 may form a closed loop. In the illustrated embodiment, the distal end of the eyelet 111 may be encapsulated in adhesives or the like to secure the eyelet 111 to a wire 101 of the embolic coil 100 or to a cap 102 secured to the embolic coil 100. However, various other methods may be utilized as discussed herein.

With reference to FIG. 8B, it can be seen that another example embodiment may utilize a pair of eyelet connections 111A, 111B which attach the distal end of the eyelet 111 to a distal region of the embolic coil 100. More specifically, FIG. 8B illustrates an example embodiment in which the eyelet 111 may comprise an open loop comprised of a substantially U-shaped configuration having a pair of distal ends. A first distal end of the eyelet 111 may be attached to a first position on the distal wind of the wire(s) 101 forming the embolic coil 100 by a first eyelet connection 111A (e.g., by being tied) and a second distal end of the eyelet 111 may be attached to a second position on the distal wind of the wire(s) 101 forming the embolic coil 100 by a second eyelet connection 111B (e.g., by being tied).

Put differently, the example embodiments shown in FIGS. 7A-8B may use a loop on the detachment monofilament tether 105A as an ‘eyelet’ 111 through which the stretch resistant monofilament tether 105A can be threaded, similar to the embodiments shown in FIGS. 3A-4B. The ‘eyelet knot’ forming an enlargement 107 may have a larger OD than the ID of the tubular member 109 once tied, so that the stretch resistant member 105 will stay within the embolic coil 100. Such example embodiments use separate (two piece) stretch and attachment monofilaments. The stretch resistant member 105 may have a smaller diameter but have two filaments running to the distal end of the embolic coil 100. The filaments of the stretch resistant member 105 may be terminated at the distal end of the embolic coil 100 using either a knot and adhesive (FIGS. 3A-3B show a single knot), melting the filaments into an atraumatic cap 102 (FIGS. 7A-7B), dual knots 111A, 111B at the distal end of the embolic coil (FIGS. 8A-8B) or a combination of all three methods).

### Clauses

Exemplary embodiments are set out in the following numbered clauses.

Clause 1. A method of manufacturing an embolic coil device may comprise tying a knot into a tether, pulling the tether through a linearized coiled wire, and securing the tether to a distal end of the linearized coiled wire.

Clause 2. A method of manufacturing an embolic coil device may comprise forming an enlargement on a tether, pulling the tether through a linearized coiled wire, and securing the tether to a distal end of the linearized coiled wire.

Clause 3. A method of manufacturing an embolic coil device may comprise attaching an enlargement to a tether, pulling the tether through a linearized coiled wire, and securing the tether to a distal end of the linearized coiled wire.

Clause 4. A method according to any of the preceding clauses may comprise encapsulating the enlargement in adhesive and securing the enlargement within the linearized coiled wire.

Clause 5. A method according to any of the preceding clauses may comprise tying a distal end of the tether to the distal end of the linearized coiled wire.

Clause 6. A method according to any of the preceding clauses may comprise attaching a filament between the distal end of the tether and the distal end of the linearized coiled wire.

Clause 7. A method according to any of the preceding clauses may comprise forming an eyelet and attaching the eyelet between the distal end of the tether and the distal end of the linearized coiled wire.

Clause 8. A method of manufacturing an embolic coil device may comprise forming a polymer filament into a braid with a web braider, inserting the braid into an interior of a linearized coiled wire, securing the braid and securing the braid within the interior of the linearized coiled wire.

Clause 9. A method according to claim 8 may further comprise pulling a tether through the interior of the linearized coiled wire and securing a distal end of the tether to a proximal end of the braid.

Clause 10. A method of manufacturing an embolic coil device may comprise forming a wavy tether by helically winding the tether around a wire or other polymer prior to assembly and then removing the extra wire or polymer after the tether has been pulled through a linearized coiled wire.

Clause 11. A method of manufacturing an embolic coil device may comprise forming a wavy tether by winding a tether over a fixture such as a mandrel at a loose pitch so that the tether has a very slightly coiled shape.

Clause 12. A method of manufacturing an embolic coil device may comprise forming a wavy tether by cutting a tether longer than a linearized coiled wire, stretching the linearized coiled wire, bonding both ends of the tether within the linearized coiled wire, and then letting the linearized coiled wire relax.

Clause 13. A method of manufacturing an embolic coil device may comprise leaving a portion of a tether intended to extend within an interior of a linearized coiled wire longer than the linearized coiled wire, connecting a distal end of the tether to a distal end of the linearized coiled wire, and pushing the extra length of the tether into the linearized coiled wire.

Clause 14. A method of delivering an embolic coil device to a target location within a body may comprise advancing a catheter through a vasculature to reach the target location, advancing a pusher through the catheter, withdrawing the catheter to expose an embolic coil, and detaching the embolic coil from the pusher.

Clause 15. A method according to clause 14 may further comprise the embolic coil resisting stretching or unwinding during delivery through use of a stretch resistant element.

Clause 16. A method according to clause 15 may further comprise the stretch resistant element being prevented from withdrawing proximally past a tubular member at a proximal end of the embolic coil by an enlargement of the stretch resistant element abutting against the tubular member.

Clause 17. A method according to any of the preceding claims may comprise an enlargement being prevented from entering an internal lumen of a tubular member attached to a proximal end of the embolic coil due to the enlargement having a greater width or diameter than the internal lumen of the tubular member.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An embolic coil device, comprising:
a coiled wire including a distal end, a proximal end, and an interior;
a tubular member attached at or near the proximal end of the coiled wire, the tubular member including an interior lumen;
a tether connected with the distal end of the coiled wire and extending through the interior of the coiled wire and the tubular member, wherein the tether comprises an enlargement; and,
wherein the enlargement is comprised of a greater width or diameter than the interior lumen of the tubular member.

2. The embolic coil device of claim 1, wherein the enlargement is not directly connected to the coiled wire and wherein at least a portion of the tether is stretch-resistant.

3. The embolic coil device of claim 1, wherein the enlargement is positioned entirely within the interior of the coiled wire.

4. The embolic coil device of claim 1, wherein the enlargement is freely movable within the interior of the coiled wire.

5. The embolic coil device of claim 1, wherein the enlargement is comprised of a ball of adhesive.

6. The embolic coil device of claim 1, wherein the enlargement is comprised of a bead fixed to the tether.

7. The embolic coil device of claim 1, wherein the tether is wavy when the tether is not under tension.

8. The embolic coil device of claim 1, wherein the tether is positioned along a central longitudinal axis of the interior of the coiled wire so as to provide on-axis torque.

9. The embolic coil device of claim 1, wherein the tether is connected to the distal end of the coiled wire by a filament.

10. The embolic coil device of claim 9, wherein the filament is tied to the distal end of the coiled wire.

11. The embolic coil device of claim 9, wherein the filament is melted against the distal end of the coiled wire to form a melted tip.

12. The embolic coil device of claim 1, further comprising a braid positioned within the interior of the coiled wire, the braid being attached to a distal end of the tether.

13. The embolic coil device of claim 12, wherein an outer diameter of the braid is less than an inner diameter of coiled wire.

14. The embolic coil device of claim 1, wherein the tether is connected to the distal end of the coiled wire by an eyelet.

15. The embolic coil device of claim 14, wherein the eyelet comprises a closed loop.

16. The embolic coil device of claim 14, wherein the eyelet comprises a substantially U-shape, and wherein a pair of distal ends of the eyelet are each tied to the coiled wire.

17. A system for delivering an embolic coil to a target location, comprising:
a delivery catheter;
a coiled wire having a distal end, a proximal end, and an interior;
a tubular member attached to the proximal end of the coiled wire, the tubular member including an interior lumen having an inner diameter which is smaller than an inner diameter of the interior of the coiled wire;
a tether extending through the delivery catheter and the tubular member, wherein a distal end of the tether is connected with the distal end of the coiled wire; and,
wherein the tether includes an enlargement having a diameter or width greater than the inner diameter of the interior lumen of the tubular member.

18. The system of claim 17, wherein the tether is connected with the distal end of the coiled wire by a filament, wherein a proximal end of the filament is attached to a distal end of the tether, and wherein a distal end of the filament is attached to the distal end of the embolic coil.

19. The system of claim 17, further comprising a braid positioned within the interior of the coiled wire, the braid being attached to a distal end of the tether.

20. An embolic coil device, comprising:
a coil means for occluding a vessel, the coil means including a distal end, a proximal end, and an interior;
a radiopaque marker band attached at or near the proximal end of the coil means, the radiopaque marker band including an interior lumen; and
a stretch resistant means for resisting stretching or unwinding of the coil means extending through the band and into the interior of the coil means, wherein the stretch resistant means comprises a stopper means for preventing the stretch resistant means from withdrawing into the radiopaque marker band.
